(19) United States
(12) Patent Application Publication
(10) Pub. No.: US 2006/0058841 A1
(43) Pub. Date: Mar. 16, 2006

(54) Connector for a hemostatic compression pad

(76) Inventors: Gary N. Mills, Gladstone, OR (US); Philip D. Benz, Portland, OR (US); Benjamin P. Mergen, Corbett, OR (US); William R. Huseby, Vancouver, WA (US)

Correspondence Address: STOEL RIVES LLP, 900 SW FIFTH AVENUE, SUITE 2600, PORTLAND, OR, 97204-1268, US

(21) Appl. No.: 10/941,292

(22) Filed: Sep. 14, 2004

(51) Int. Cl. A61B 17/00

(52) U.S. Cl. 606/201

(57) ABSTRACT

A machine comprises a compression apparatus and a compression pad. The compression apparatus is capable of generating pressure onto an area of a subject's body. The compression apparatus comprises a cavity having an interior sidewall. The compression pad is disposed between the compression apparatus and the area of the subject's body. The pressure is applied onto the area of the subject's body through the compression pad. The compression pad has a first side and a second side. The second side is intended to be oriented toward the area of the subject's body. The first side is intended to connect to the compression apparatus. A post is located on the first side of the compression pad. The post has an exterior sidewall. The post fits into the cavity, contacts the interior sidewall of the cavity, and is retained in the cavity by friction.

### TECHNICAL FIELD

This disclosure relates generally to surgical instrumentation and more particularly to external pressure application.

### BACKGROUND

The femoral artery is one example of a high pressure blood vessel that requires deliberate action to achieve hemostasis (cessation of bleeding) following completion of a cannulation procedure. If a sheath is removed from, for example, the femoral artery in the groin and no attempt to achieve hemostasis is made, the patient would quickly experience rapid bleeding resulting in adverse health consequences including hypovolemia, shock, a requirement for blood transfusion, and possibly death. Application of pressure proximal and medial to a femoral artery puncture site and directly over the femoral artery achieves hemostasis whenever the pressure applied is sufficient to slow or completely occlude blood flow in the femoral artery. This permits a clot to form and thereby results in hemostasis at the puncture site.

One technique of applying such pressure requires that a practitioner remain with the patient and actively press down with gloved hands directly over the patient's artery and generally proximal and medial to the puncture site. The duration of application of pressure varies based on the type of procedure, the nature of the drugs administered, and the patient's condition. This period of time frequently extends between 15 and 30 minutes, or longer. As a result, fatigue, stiffness, and pain may occur in the fingers, hands, wrist, and forearms of the practitioner performing this procedure. A practitioner repeatedly performing this procedure over a long period of time without the aid of any assist devices could develop repetitive strain injury, for example, carpal tunnel syndrome. Also, there is the possibility that a glove could have or develop a hole, thereby allowing direct pressurized skin contact with a patient's blood.

A number of possible solutions to these problems may be collectively known as vascular compression devices, which are described in the prior art. With these solutions, a compression pad is used in conjunction with a vascular compression apparatus to compress a portion of a patient's body surface, in turn compressing a blood vessel and subsequently resulting in hemostasis. In most vascular compression devices, a removable compression pad is attached to and detached from the compression apparatus applying the compression force by means of a connector.

One possible solution is described by Semler in U.S. Pat. Nos. 3,779,249 and 5,304,186. The devices described in those two patents are generally comprised of a c-clamp mechanism, to which a detachable, disk-shaped pressure pad is attached when being used on a patient. Although the c-clamp mechanism is advantageous in that it frees the hands of the attending practitioner and provides consistent pressure during the compression period, the attachment of the pressure pad to the mechanism is not always stable because the pressure pad is attached to the mechanism by a simple friction fit.

Commonly used compression pads, which include some of those described in the prior art, are made of generally rigid materials, for example, a plastic such as an acrylic. Connectors, for the purpose of removably attaching a compression pad to a compression device, may be one of a ball-and-socket type, a keyhole aperture, or a friction fit. In each of these connectors, a female portion of the connector is located on the pad and a male portion is located on the vascular compression apparatus. Another type of connector utilizes an adhering material, for example, VELCRO or a hook-and-loop material.

Of the known prior art connectors, only a ball-and-socket connector provides a positive retention of the pad to the compression apparatus; however, because of the construction of such devices, attachment and removal of the pad requires unnecessary effort. The remaining types of connectors permit the pad to fall from the compression apparatus accidentally, since there is no mechanism for or material positively retaining the pad to the apparatus.

Patient body contours can often cause uneven distribution of pressure across the bottom surface of a compression pad, for example, when the compression pad is applied under pressure to a patient having a large abdomen. In this instance, pressure can be very high on the proximal portion of the compression pad and very low or non-existent on the distal portion; in some cases, the distal portion may not make contact with the body surface. Such uneven pressure distribution can cause unwanted deflection of the compression pad, sometimes resulting in fracture of one or more areas of the pad, including the receptacle on the pad since the only area of contact between the compression pad and the compression apparatus is through the post on the apparatus and the receptacle on the pad.

In summary, disadvantages of prior art connectors, including devices in commercial use, include: (1) accidental detachment of the compression pad from the compression apparatus; (2) excessive deformation of the compression pad leading to fracture of the compression pad when under pressure, due to compression pressures inadequately supported across the structure of the compression pad; and (3) deformation of the connector, leading to inadequate stability of the compression point on the body surface when the compression pad is under pressure, due to the structure of the male post connector element on the compression apparatus.

### SUMMARY

The invention provides an improved connection of a compression pad to a compression apparatus.

In one respect, the invention is a machine comprising a compression apparatus and a compression pad. The compression apparatus is capable of generating pressure onto an area of a subject's body. The compression apparatus comprises a cavity having an interior sidewall. The compression pad is disposed between the compression apparatus and the area of the subject's body. The pressure is applied onto the area of the subject's body through the compression pad. The compression pad has a first side and a second side. The second side is intended to be oriented toward the area of the subject's body. The first side is intended to connect to the compression apparatus. A post is located on the first side of the compression pad. The post has an exterior sidewall. The post fits into the cavity, contacts the interior sidewall of the cavity, and is retained in the cavity by friction.

In another respect, the invention is a compression pad capable of use with a compression apparatus that applies pressure onto an area of a patient's body through the compression pad. The compression apparatus comprises a cavity located where the compression pad attaches to the compression apparatus. The cavity has an interior sidewall. The compression pad comprises a first side and a second side. On the first side is located a post intended to connect to the compression apparatus. The post fits into the cavity. The post has an exterior sidewall, which contacts the interior sidewall of the cavity. The post is retained in the cavity by friction. The second side is intended to be oriented toward the area of the patient's body.

In yet another respect, the invention is a method for removably attaching a compression pad to a compression apparatus capable of applying pressure onto a target area of a patient's body through the compression pad. The compression pad has opposing first and second sides. The second side is intended to be oriented toward the target area of the patient's body. The first side comprises a post having an exterior sidewall. The compression apparatus comprises a cavity having an interior sidewall. The method fits the post within the cavity, whereby the exterior sidewall of the post contacts the interior sidewall of the cavity and is retained therein by friction. The method, places the second side of the compression pad against the target area of the patient's body. The method applies pressure from the compression apparatus through the cavity against the post of the compression pad, thereby causing the second side of the compression pad to press against the target area of the patient's body.

Additional details concerning the construction and operation of particular embodiments of the invention are set forth in the following sections with reference to the below-listed drawings.

### BRIEF DESCRIPTION OF THE DRAWINGS

FIG. 1 is partial side section view of a hemostatic compression pad applied to a body surface with a compression apparatus, according to one embodiment.

FIG. 2 is a perspective view of a hemostatic compression pad, according to one embodiment.

FIGS. 3A and 3B are perspective views of the components of the hemostatic compression pad of FIG. 2.

FIGS. 4A and 4B are top plan views of the components of the hemostatic compression pad of FIG. 2.

FIG. 5 is a bottom plan view of the hemostatic compression pad of FIG. 2.

FIG. 6 is a perspective view of an arm slide component of a compression apparatus, according to one embodiment.

FIG. 7 is a bottom plan view of the arm slide of FIG. 6.

FIG. 8 is a perspective view of an arm slide and hemostatic compression pad joined together, according to one embodiment.

FIG. 9 is a side cut-away view of the arm slide and hemostatic compression pad joined together, taken from FIG. 8.

FIG. 10 is a flowchart of a method of attaching a compression pad to a compression apparatus, according to one embodiment.

FIG. 11 is a side view of an adaptor according to another embodiment.

### DETAILED DESCRIPTION OF PREFERRED EMBODIMENTS

### A. Overview

The embodiments described herein assist a user in controlling bleeding and achieving hemostasis through the stable application of pressure onto a subject's body surface thereby compressing the lumen of an underlying blood vessel, in turn reducing or halting blood flow through said blood vessel. More particularly, certain embodiments are capable of achieving certain advantages, including some or all of the following: (a) enabling more stable compression; (b) increasing durability of the compression pad when placed under pressure onto a body surface; (c) improving attachment of the compression pad to the compression apparatus and subsequent orientation and detachment; and (d) avoiding accidental detachment of the pad from the compression apparatus. These and other advantages will be apparent from the following.

The general structure of the connector includes a post located on a compression pad, and a receptacle located on a compression apparatus. The post fits into the receptacle such that friction retains the compression pad on the compression apparatus; the interior contour of the receptacle generally mates to the exterior contour of the post. At least a portion of the compression pad and the connector is preferably composed of a generally rigid first material, for example a plastic.

Use of textures or materials on the surfaces of the post and receptacle can also affect the nature of the friction fit retaining the compression pad to the compression apparatus. The compression pad includes a generally rigid first material composing a substrate, and may, optionally, further include a generally resilient, compressible second material composing other portions of the compression pad. The second material, for example containing polyurethane or silicone or thermoplastic elastomer, and having the general properties of resiliency, compressibility, pliancy and a higher coefficient of friction than the first material, may be included on at least a portion of the post or receptacle, or compression pad, in order to improve the retention created by the friction fit without necessarily using a material having the property of adhesiveness.

In a preferred embodiment, the second material fits onto or within portions of the compression pad, including the post and, optionally, at least a portion of the bottom surface of the compression pad. More particularly, the second material may take the form of an insert, which would fit into a hollow space within the post of the compression pad, and include at least one protuberance or flute protruding out from the side of the portion of the insert that fits into the hollow space. The flutes or protuberances fit into slots located on a vertical sidewall of the post, such that they extend past the exterior surface of the sidewall to make contact with the interior surface of the receptacle before the sidewall of the post does. Alternatively or additionally, the second material can be included on the interior sidewall of the receptacle. Use of the second material, in particular, enables improved attachment, retention and detachment, in part due to a resultant graduated friction fit, as shall be explained elsewhere with reference to the drawings.

The receptacle element of the compression apparatus includes an opening through which the post is inserted when attaching the compression pad, which has a top surface. A rim surrounds this opening. When the compression pad is applied under pressure to the body surface, the horizontal plane of the compression pad deflects in the vertical axis. The top surface of the compression pad will make contact with the rim only during such deflection, limiting the degree of deflection and providing an additional area of support for the compression pad under pressure, and therefore limiting the amount of flex caused by such application. Since deflections of a severe nature can fracture the compression pad, limiting such deflection and distributing the pressure across a greater surface of the compression pad and compression apparatus improves durability of the compression pad by decreasing the opportunity for fracture. By limiting the extent of the deflection, the rim's contact with the compression pad's top surface also helps provide more stable compression.

More stable compression and increased compression pad durability result from the stronger structure provided by the male post on the compression apparatus, and by having contact between the compression pad and the compression apparatus occur not only between the exterior sidewall of the compression pad post and interior sidewall of the compression apparatus receptacle, but also by having contact made, when the compression pad is placed under pressure onto a body surface, between the top surface of the compression pad and rim of the receptacle when the compression pad flexes under such pressure. Improved attachment and detachment, and retention of the post of the compression pad in the receptacle of the compression apparatus, reduces the opportunity for the compression pad to accidentally detach from the compression apparatus, due to the higher coefficient of friction between the elastomeric component of the compression pad and the interior surface of the receptacle and the resultant graduated friction fit created between the elastomeric component of the compression pad and the interior surface of the receptacle, while still permitting the user to easily rotate the compression pad within the receptacle to achieve proper orientation relative to the puncture site.

Many prior art compression apparatuses and the associated removable, generally disposable prior art compression pads have been in use for at least several years as of the date of this application. The inventors therefore contemplate a need for compatibility with prior art compression pads, where the compression apparatus embodies the new connector, in order to transition use from the currently-used compression pads to the compression pad described herein, thereby avoiding waste of compression pads already possessed by a user, or alternatively to enable a user to easily use both the compression pad described herein and the prior art compression pads. An easy means of providing economical access to the improved connectors described herein is through the use of an adaptor which could permit such use.

In a preferred embodiment, the adaptor is composed of a generally rigid material, which may be inexpensively molded, machined or constructed. As a structure having two ends which may be generally opposed, it includes at one end a post to connect to a receptacle on a compression apparatus, and at the other end a connection means suitable for attaching compression pads having other connection means. The post fits into the receptacle such that friction retains the adaptor on the compression apparatus; the interior contour of the receptacle generally mates to the exterior contour of the post which generally has the characteristics of the connector in the foregoing description. The adaptor therefore connects both to a prior art compression pad and the compression apparatus described herein.

Such an adaptor can achieve some or all of the following benefits: (a) prior art compression pads may be used with a compression apparatus employing the connector described in this invention, thereby avoiding waste of such prior art compression pads that the user may already possess; (b) a user is able to use both prior art compression pads and the compression pad described herein on the same compression apparatus described herein; and (c) the adaptor is inexpensive.

### B. Illustrated Embodiments

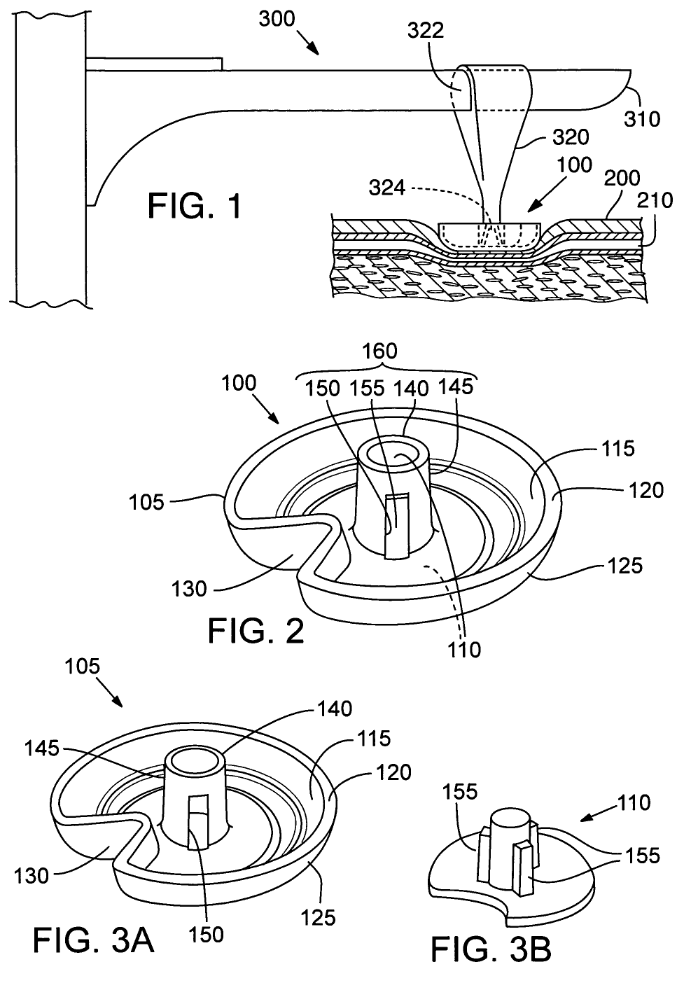

FIG. 1 is partial side section view of a hemostatic compression pad 100 applied to a body surface 200 with a compression apparatus 300, according to one embodiment. The body surface 200 is generally over a blood vessel 210 for which control of bleeding and hemostasis is desired. In one foreseeable use, the blood vessel 210 is the femoral artery in the groin. As previously explained, that vessel requires deliberate action to achieve hemostasis following a cannulation procedure. The compression pad 100, in cooperation with the compression apparatus 300, depresses the area of the body surface 200 upon which it is placed, thereby compressing the lumen of the blood vessel 210 over which it is placed to partially or completely occlude the blood vessel 210. Such occlusion limits or completely stops the flow of blood in the blood vessel 210 during such application and maintenance of the pressure, enabling hemostasis to occur. Once hemostasis occurs the compression apparatus 300 is loosened and the compression pad 100 is carefully removed from the patient so as to preserve the hemostasis of the puncture site. The patient or subject of the compression is typically a human, although that need not be the case. The compression pad 100, with the compression apparatus 300, may also be used to externally compress other bodily tissue for other purposes.

The compression apparatus 300 comprises an arm 310 on which an arm slide 320 is attached. The compression apparatus 300 preferably applies force onto the body surface 200 through the compression pad 100 by mechanical means. Moreover, the compression apparatus 300 maintains that force mechanically with no human involvement. One example of a compression apparatus 300 suitable for use with the compression pad 100 is disclosed in commonly owned U.S. patent application Ser. No. 10/011,828, filed Nov. 30, 2001, which is herein incorporated by reference.

The arm 310 extends in a direction transverse (horizontally, as shown in FIG. 1) to the direction at which pressure is applied (vertically, as shown in FIG. 1) to the body surface 200. The arm slide 320 preferably comprises two ends, as shown. At the top end is a chute 322 extending through the arm slide 320. The chute 322 is a tunnel whose interior shape and dimensions are such that the chute 322 snugly and slidably fits over the arm 310 in a mating configuration. The position of the arm slide 320 along the arm 310 is preferably adjustable.

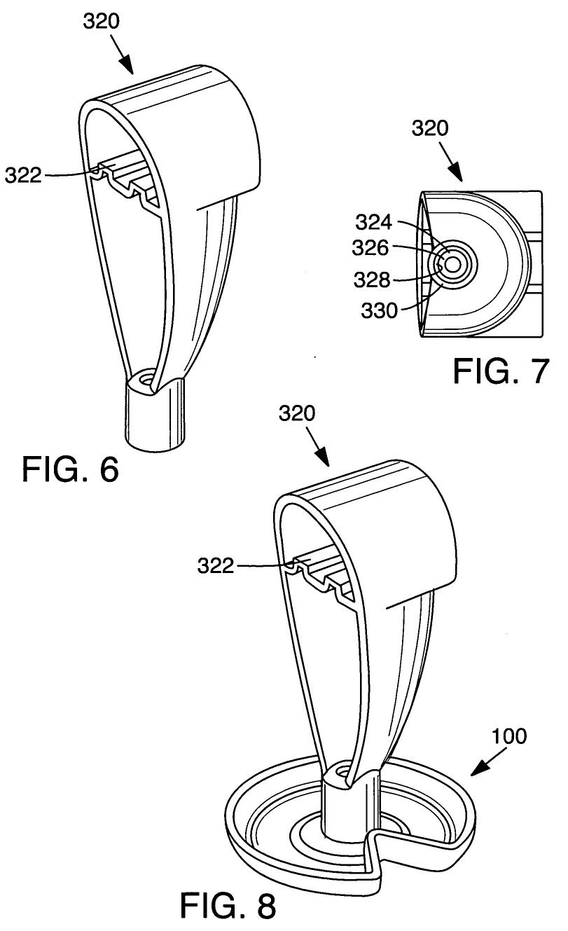

At the bottom end of the arm slide 320 is a cavity 324, where the arm slide 320 and the compression pad 100 connect. The opening of the cavity 324 is along the bottom of the arm slide 320, and the cavity 324 extends upward within the body of the arm slide 320. The cavity 324 has an interior sidewall, which is preferably frustoconical or tapered in shape, and an optional ceiling. The interior sidewall may be in a different shape, such as cylindrical, columnar, or conical, for example. Although reference is made to an arm slide 320 as an element separate from the arm 310, the cavity 324 and its supporting structures may be optionally included on the arm 310, thereby obviating the need for a separate arm slide 320. Additional details about one exemplary arm slide 320 are presented below with reference to FIGS. 6-8.

Although the terms “top,” “bottom,” and “ceiling” have been used to describe the parts of FIG. 1, it should be understood that those terms are used to facilitate understanding of the illustrated embodiments, not to imply that such a directional orientation is necessary. The arrangement of FIG. 1 may be oriented in any direction, such as the opposite direction (applying pressure upwards onto the body surface 200) or sideways.

FIG. 2 is a perspective view of the hemostatic compression pad 100, according to one embodiment. The compression pad 100 includes a substrate 105 and an insert 110. The substrate 105 is preferably composed of a first, generally rigid material, such as a polycarbonate, a polypropylene, an acrylic, or other plastic, for example. The insert 110 is preferably composed of a second material having at least one of the properties of compressibility, resilience, elasticity, pliancy, non-adhesiveness, and a higher coefficient of friction than the material composing the substrate 105. For example, the second material can include polyurethane, silicone, elastomeric thermoplastic, or olefin.

The substrate 105 includes a top side 115, a rim 120, and a bottom side 125. The top side 115 has a generally concave curvature near the rim 120, and the bottom side 125 is generally convex near the rim 120. Thus, the rim 120 is generally turned upward. The shape of the circumference of the compression pad 100 is generally defined by the rim 120, which is substantially circular with a notch 130, which may be in a V shape, with the widest portion of said V shape at the perimeter, and with the apex pointed towards the center of the compression pad 100. The notch 130, though optional, permits the compression pad 100 to be placed near a puncture site while a catheter is in the blood vessel 210. In that case, the portion of the catheter protruding from the puncture site fits within the notch 130. Alternative shapes and/or sizes of the circumference and of the notch 130 are possible.

On the top side 115 of the substrate 105 is a post stub 140, which extends upward from the top side 115. The post stub 140 has an exterior sidewall 145, which is preferably in a frustoconical or tapered shape, with the wider portion at the base near the surface of the top side 115, and the narrower portion at the top end of the post stub 140. Other shapes, for example cylindrical, columnar or conical, are also possible for the sidewall 145. The post stub 140 of the substrate 105 is preferably hollow and has one or more openings 150, along the sidewall 145 from the top side 115 to the hollow interior of the post stub 140.

In a preferred embodiment, the insert 110 attaches to the substrate 105, whereby the insert 110 covers at least a portion of the substrate's bottom side 125 and extends into the hollow portion of the substrate's post stub 140. The insert 110 preferably includes one or more protuberances 155 that fit into the openings 150 on the post stub 140. The protuberances 155 can be any flutes, ribs, protrusions, or keys fitting into the keyways formed by the openings 150. Because the material of the insert 110 preferably has the properties of compressibility, resilience, pliancy, elasticity, and a greater coefficient of friction (without necessarily having a property of adhesiveness), it is advantageous that a portion of the material of the protuberances 155 is exposed on the exterior sidewall 145 of the post stub 140 and preferably extends past the sidewall 145 of the post stub 140. A portion of the insert 110 may extend out of and above the top open end of the post stub 140. Optionally, that upwardly extending portion of the insert 110, if present initially, can be later removed.

The post stub 140 of the substrate 105 and the portion of the insert 110 that extends into the hollow portion of the post stub 140, as well as the protuberances 155 that extend through the openings 150 and any portion of the insert 110 extending above the post stub 140, together constitute a post 160 of the compression pad 100. The post 160 is an example of the compression pad 100's means for removably attaching to the compression apparatus 300, in particular to the arm slide 320.

The substrate 105 and the insert 110 are preferably separate pieces, as shown in the perspective views of FIGS. 3A and 3B respectively. The substrate 105 is an outer shell that mostly surrounds the insert 110, which is an interior portion. When the two pieces are attached, the insert 110 is retained within the substrate 105 by friction and/or preferably by the protuberances 155 protruding through the openings 150. Alternatively, the substrate 105 and the insert 110 may be an integral, non-separable item. For example, the insert 110 may be molded in the substrate 105, adhered to the substrate 105, or otherwise attached to the substrate 105 using techniques known in the art.

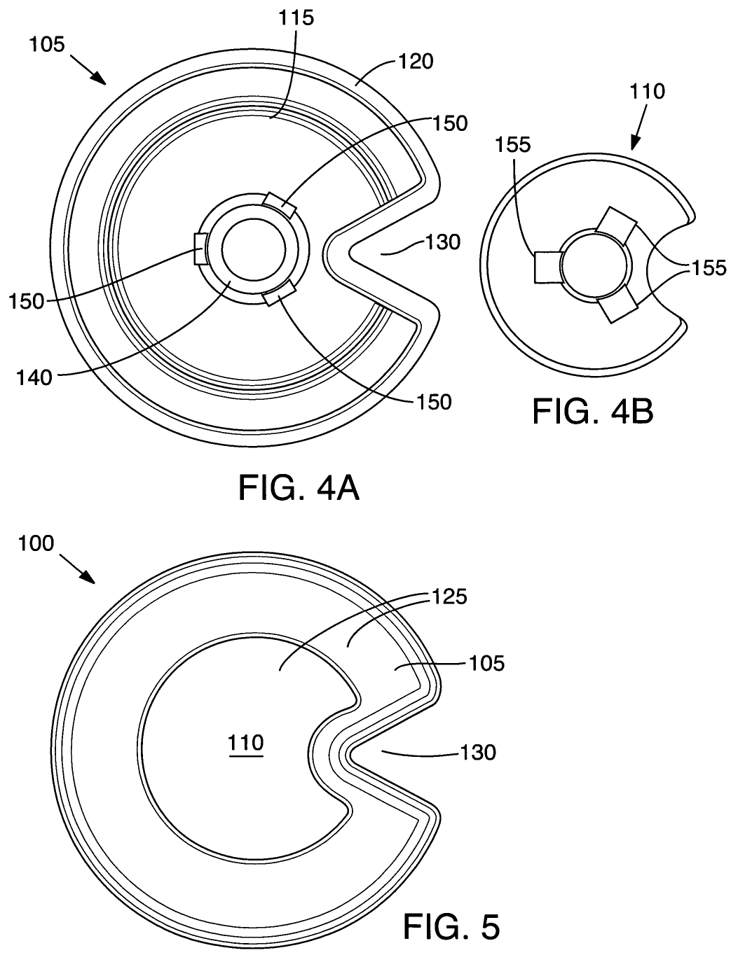

FIGS. 4A and 4B are top plan views of the substrate 105 and the insert 110, respectively. FIGS. 4A and 4B illustrate the substrate 105 with three openings 150 equally spaced around the post portion 150, and three matching protuberances 155 on the insert 110. A lesser or greater number of openings 150 and protuberances 155 are possible.

FIG. 5 is a bottom plan view of the compression pad 100 formed of the substrate 105 and the insert 110 together. FIG. 5 illustrates that the bottom side 125 of the compression pad 100 at least partially consists of the same second material composing the insert 110. Though optional, that arrangement is advantageous because the second material, which preferably has a higher coefficient of friction against skin (compared to the material composing the substrate 105) is less likely to slip laterally across the patient's body surface 200 during use.

FIG. 6 is a perspective view of the arm slide 320, according to one embodiment. As this drawing shows, the chute 322 contains ridges and grooves along its interior bottom sidewall, which further has a shape snugly and slidably fitting and mating onto the arm 310 of the compression apparatus 300, where said shape is generally convex on its top interior side. The convex shape of the top sidewall of the chute 322 results in increased strength and stiffness, leading to decreased deflection and permitting a thinner wall section (requiring less material and thus resulting in a lighter weight and less cost). Though optional, the ridges and grooves are useful in preventing the arm slide 320 from twisting or pivoting around the arm 310, which has complementary, mating valleys and protrusions.

FIG. 7 is a bottom plan view of the arm slide 320 of FIG. 6. FIG. 7 reveals the cavity 324 from its open end. The cavity 324 is a receptacle that connects with the post 160 of the compression pad 100. The opening of the cavity 324 is in a generally circular shape, like the post 160 of the compression pad 100; however, the opening may be in any shape provided it is capable of receiving the post 160. The cavity 324 has an optional ceiling 326 and an interior sidewall 328. The interior sidewall 328 optionally has an irregular or roughened surface texture (e.g., ridges or dot-like bumps) to enhance gripping of the post 160 on the compression pad 100. Alternatively or additionally, at least a portion of the interior sidewall 328 may optionally include a second material having at least one of the properties of compressibility, resilience, elasticity, pliancy non-adhesiveness and a higher coefficient of friction. The second material enables a graduated friction fit when a mating post of a compression pad is inserted for the purpose of attachment. Along the opening of the cavity 326 is a rim 330. The rim 330 is the bottommost portion of the arm slide 320. As previously mentioned, terms such as “top,” “bottom,” and “ceiling” are used herein as relative terms to facilitate understanding of the illustrated embodiments, not to preclude uses of the invention in other directional orientations.

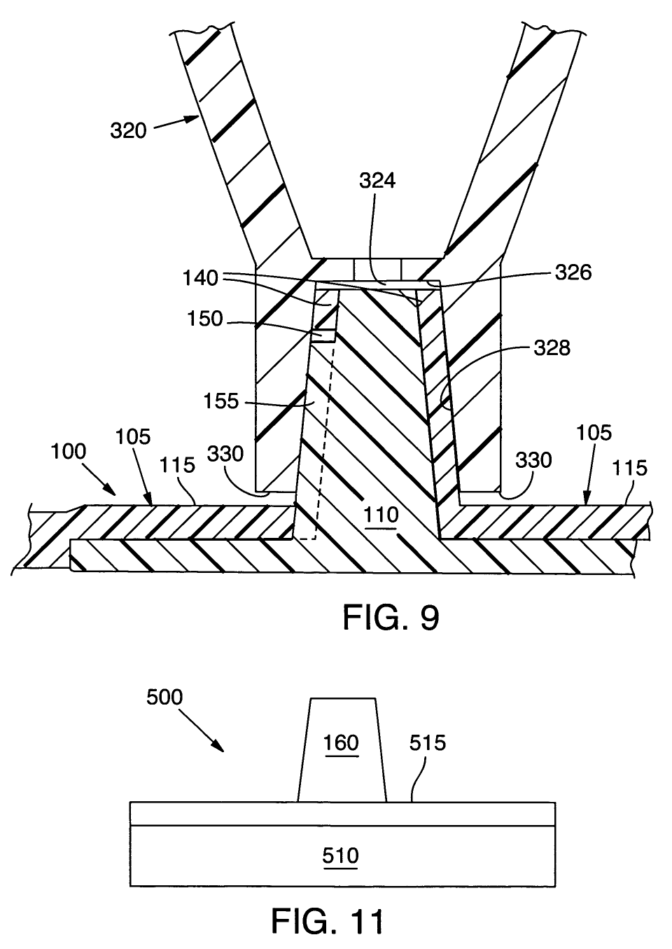

FIG. 8 is a perspective view of the arm slide 320 and the compression pad 100 joined together, according to one embodiment, and FIG. 9 is a cut-away side view taken from FIG. 8 where the arm slide 320 and the compression pad 100 connect. As FIG. 9 shows, the protuberances 155 of the insert 110 extend through the openings 150 of the post stub 140 and contact the interior sidewall 328 of the cavity 324 of the arm slide 320. That arrangement results in a graduated fit of the post 160 in the cavity 324.

In particular, use of a second material for the insert 110 enables a graduated friction fit whereby the compression pad 100 is retained on the compression apparatus 300 in three graduated phases: First, the post 160 is not retained in the cavity 324, and the compression pad 100 may therefore freely detach altogether from the compression apparatus 300. Second, the post 160 is retained in the cavity 324 by means of the contact made by the second material, of the protuberances 155, with the surface of the interior sidewall 328 of the cavity 324, and because the fit is not snug, the user may rotate the compression pad 100 while it is attached to the compression apparatus 300 without the compression pad 100 freely detaching from the compression apparatus 300. Third, the post 160 is snugly fit in the cavity 324 through the compression of the protuberances 155 that is caused by its contact, under pressure, with the interior surface of the cavity 324, and by the post 160 in contact under pressure with the interior surface of the cavity 324, causing retention on the compression pad 100 to be secure; the compression pad 100 therefore may be rotated, if at all, only with additional exertion by the user.

The material of which the insert 110 and thus the protuberances 155 are made preferably has at least one of the properties of compressibility, resilience, elasticity, pliancy, and a higher coefficient of friction than the material of which the substrate 105 is made. Different shapes of the post 140 and/or the cavity 324 as well as the use of other materials or textures on the surfaces of the exterior sidewall 145 of the post 160 and/or the surface of the interior sidewall 328 of the cavity 324 can also affect the nature of the graduated friction fit retaining the compression pad 100 to the compression apparatus 300. A roughened texture or irregular pattern of features, for example ridges or dots, on either or both of these surfaces will increase the coefficient of friction between the surfaces, thereby increasing the degree of retention of the compression pad 100 on the compression apparatus 300, and optionally obviating the need for the post stub 140 to be in contact with the interior surface of the cavity 324 to ensure secure retention.

When the compression pad 100 is applied under pressure to the body surface 200, the horizontal plane of the compression pad 100 deflects in the vertical axis. The top surface 115 of the compression pad 100 will make contact with the rim 330 during such deflection, limiting the degree of deflection, and therefore limiting the amount of flex caused by such application. Since deflections of a severe nature can fracture the compression pad 100, limiting such deflection and distributing the pressure across a greater surface of the compression pad 100 improves durability of the compression pad 100 by decreasing the opportunity for fracture. By limiting the extent of the deflection, the contact of the rim 330 with the top surface 115 of the compression pad 100 also helps provide more stable compression.

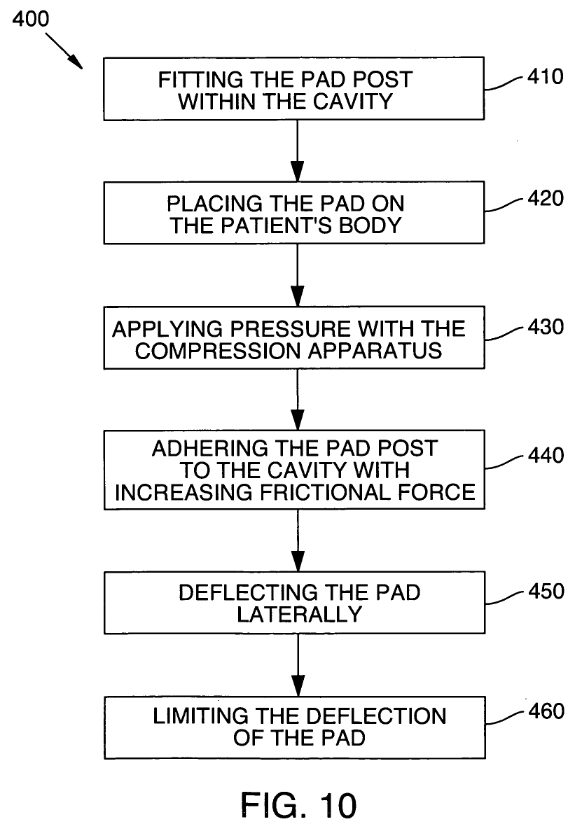

FIG. 10 is a flowchart of a compression method 400, according to one embodiment. The method 400 begins by fitting (410) the post of the compression pad 100 within the cavity 324 of the compression apparatus 300. At this point, because the post is only loosely attached to the cavity, the compression pad 100 may be rotated about the vertical axis of the post to a desired angular position. Next, the method 400 places (420) the compression pad 100 on the patient's body surface (200). Thereafter, the method 400 applies (430) pressure with the compression apparatus 300. The pressure application step 430 is preferably performed with minimal human intervention to initially apply the pressure, which is thereafter mechanically maintained without the need for constant human intervention. As a result of the pressure application step 430, the method adheres (440) the post of the compression pad 100 to the cavity 324 with increased frictional force. Also as a result of the pressure application step 430, the method might deflect (450) the compression pad laterally. However, if that occurs, then the method limits (460) that deflection, preferably by contacting the rim 330 of the compression apparatus against the top side 115 surface of the compression pad 100.

An alternative embodiment is illustrated in FIG. 11, which depicts an adaptor 500 to enable compatibility with compression pads other than the compression pad 100. More particularly, the adaptor 500 may be useful at least during a transition period from use from those currently-used prior art compression pads to use of the compression pad 100 described herein, thereby avoiding waste of pads already possessed by a user. Alternatively, the adaptor 500 can enable a user to easily use both the compression pad 100 described in detail herein and prior art compression pads, or other, compression pads. An easy means of providing economical access to the improved connector is through the use of the adaptor 500, which could permit such use.

The adaptor 500 is preferably composed of a generally rigid material, for example an acrylic or nylon or polypropylene or polycarbonate or other plastic, which may be inexpensively molded, constructed, or machined. The adaptor 500 is a structure having two sides, which may be opposed. On a first, bottom side 510, the adaptor 500 attaches to a compression pad having connection characteristics different from those described in the foregoing description. The manner and arrangement of that attachment will vary depending upon the particular connection characteristics of the compression pad to be attached, which may include prior art connectors. Examples of prior art connectors include those with (female) receptacles intended for receiving (male) posts on a compression apparatus by a frictional fit, such as described by Royse in U.S. Pat. No. 4,572,182; ball-and-socket connectors, such as described by Freund, et al., in U.S. Pat. No. 4,742,825 or Rice in U.S. Pat. No. 5,304,201; and a keyhole type connector, such as described by Lam in U.S. Pat. No. 5,601,596. Suitable connection characteristics for connecting the first side 510 to those and other compression pads will be apparent to those skilled in the art.

On the second, opposite, top side 515 of the adaptor 500 is a post 160 generally having the characteristics attributed to the one on the compression pad 100 described above. The second side 515 of the adaptor 500 connects to the compression apparatus 300, or more particularly the arm slide 320, in the manner described above.

The terms and descriptions used herein are set forth by way of illustration only and are not meant as limitations. Those skilled in the art will recognize that many variations can be made to the details of the above-described embodiments without departing from the underlying principles of the invention. The scope of the invention should therefore be determined only by the following claims (and their equivalents) in which all terms are to be understood in their broadest reasonable sense unless otherwise indicated.

1. A machine comprising:
a compression apparatus capable of generating pressure onto an area of a subject's body, the compression apparatus comprising a cavity having an interior sidewall; and
a compression pad disposed between the compression apparatus and the area of the subject's body, the pressure being applied onto the area of the subject's body through the compression pad, the compression pad having a first side and a second side, the second side intended to be oriented toward the area of the subject's body, the first side intended to connect to the compression apparatus, the compression pad comprising a post located on the first side of the compression pad, the post having an exterior sidewall, whereby the post fits into the cavity, contacts the interior sidewall of the cavity, and is retained in the cavity by friction.

2. The machine of claim 1, wherein the shape of the exterior sidewall of the post and the shape of the interior sidewall of the cavity are approximately frustoconical.

3. The machine of claim 1, wherein the compression apparatus is capable of mechanically maintaining the pressure.

4. The machine of claim 1, wherein the frictional retention becomes stronger as the post extends further into the cavity.

5. The machine of claim 1, wherein the post comprises a first portion made of a first material and a second portion made of a second material, the second material having a higher coefficient of friction than the first material.

6. The machine of claim 1, wherein the post comprises an outer shell made of a first material and an interior portion made of a second material, the outer shell having one or more sidewall openings, and the interior portion having one or more protuberances, the one or more openings and protuberances cooperating such that the one or more protuberances fit into and extend through the one or more openings.

7. The machine of claim 6, wherein the interior portion is separable from the outer shell.

8. The machine of claim 6, wherein the outer shell is made of a generally rigid first material, and the interior portion is made of a second material having at least one of the properties of compressibility, resilience, elasticity, pliancy, and a higher coefficient of friction than the first material.

9. The machine of claim 6, wherein at least a portion of the second side of the compression pad is made of the second material and integrally connected to the interior portion of the post.

10. The machine of claim 1, wherein the cavity has an open end and a rim along the open end, and the post extends from a base on the first side of the compression pad, wherein contact between the rim of the cavity and the base of the compression pad limits deflection of the compression pad when sufficient pressure is applied to the patient's body through the compression pad.

11. The machine of claim 1, wherein the compression apparatus further comprises:
an arm extending generally perpendicular to the direction in which the pressure is applied to the area of the subject's body; and
an arm slide having two ends, the cavity being disposed on a first end, the second end comprising a chute shaped and dimensioned to fit snugly and slidably over the arm.

12. The machine of claim 1, wherein the surface of the exterior sidewall of the post has a roughened or patterned texture.

13. The machine of claim 1, wherein the surface of the interior sidewall of the cavity has a roughened or patterned texture.

14. A compression pad capable of use with a compression apparatus that applies pressure onto an area of a patient's body through the compression pad, the compression apparatus comprising a cavity located where the compression pad attaches to the compression apparatus, the cavity having an interior sidewall, the compression pad comprising:
a first side on which is located a post intended to connect to the compression apparatus, the post having an exterior sidewall, wherein the post fits into the cavity, the exterior sidewall of the post contacts the interior sidewall of the cavity, and the post is retained in the cavity by friction; and
a second side intended to be oriented toward the area of the patient's body.

15. The compression pad of claim 14, wherein the shape of the exterior sidewall of the post is approximately frustoconical.

16. The compression pad of claim 14, wherein the frictional retention becomes stronger as the post extends further into the cavity.

17. The compression pad of claim 14, wherein at least a portion of the post is made of a second material having a higher coefficient of friction than a first material composing the rest of the post.

18. The compression pad of claim 17 wherein at least a portion of the post is composed of a first material that is generally rigid and at least a portion of the post is made of a second material having at least one of the general properties of compressibility, resilience, elasticity, and pliancy.

19. The compression pad of claim 18, wherein the post comprises a substrate made of the first material and an insert made of the second material, the substrate having one or more sidewall openings, and the insert having one or more protuberances, the one or more openings and protuberances cooperating such that the one or more protuberances fit into and extend through the one or more openings.

20. The compression pad of claim 19, wherein the insert is separable from the substrate.

21. The compression pad of claim 19, wherein the insert constitutes at least a portion of the surface of the second side of the compression pad.

22. The compression pad of claim 14, wherein the surface of the exterior sidewall of the post has a roughened or patterned texture.

23. A method for removably attaching a compression pad to a compression apparatus capable of applying pressure onto a target area of a patient's body through the compression pad, the compression pad having opposing first and second sides, the second side intended to be oriented toward the target area of the patient's body, the first side comprising a post having an exterior sidewall, the compression apparatus comprising a cavity having an interior sidewall, the method comprising:
fitting the post within the cavity, whereby the exterior sidewall of the post contacts the interior sidewall of the cavity and is retained therein by friction;
placing the second side of the compression pad against the target area of the patient's body; and
applying pressure from the compression apparatus through the cavity against the post of the compression pad, thereby causing the second side of the compression pad to press against the target area of the patient's body.

24. The method of claim 23, wherein the step of applying pressure comprises:
extending the post further into the cavity, thereby causing the exterior sidewall of the post to be increasingly frictionally adhered to the interior sidewall of the cavity.

25. The method of claim 23, further comprising:
mechanically maintaining the pressure.

26. The method of claim 23, wherein the cavity has an open end and a rim along the open end, and the post extends from a base on the first surface of the compression pad, the step of applying pressure comprises:
deflecting the compression pad in a direction perpendicular to a plane along which the pressure is applied to the patient's body, as the pressure increases; and
abutting the rim of the cavity against the base of the compression pad, so as to limit the deflection of the compression pad.
